(19) United States
(12) Patent Application Publication
(10) Pub. No.: US 2020/0071651 A1
(43) Pub. Date: Mar. 5, 2020

(54) SEPARATION SUBSTRATE, CELL SEPARATION FILTER, AND METHOD FOR PRODUCING PLATELET

(71) Applicant: FUJIFILM Corporation, Tokyo (JP)

(72) Inventors: Toshiki TAKEI, Ashigara-kami-gun (JP); Tadanori YAMADA, Ashigara-kami-gun (JP); Ryuta TAKEGAMI, Ashigara-kami-gun (JP); Kuniyuki KAMINAGA, Ashigara-kami-gun (JP)

(73) Assignee: FUJIFILM Corporation, Tokyo (JP)

(21) Appl. No.: 16/674,613

(22) Filed: Nov. 5, 2019

Related U.S. Application Data
(63) Continuation of application No. PCT/JP2018/015926, filed on Apr. 18, 2018.

(30) Foreign Application Priority Data
May 12, 2017 (JP) 2017-095834

(51) Int. Cl. C12M 1/00; B01D 71/34; A61K 35/19; A61P 7/04; C12N 5/078; B01D 71/68; B01D 67/00

(52) U.S. Cl. CPC C12M 29/04; B01D 71/34; A61K 35/19; B01D 2325/02; C12N 5/0644; B01D 71/68; B01D 67/0013; A61P 7/04

(57) ABSTRACT

An object of the present invention is to provide a separation substrate having a high megakaryocyte blocking rate and a high platelet permeation rate, and a cell separation filter and a method for producing a platelet which use the same. The separation substrate of the present invention is a separation substrate including a porous membrane for separating a platelet from a cell suspension containing a megakaryocyte and the platelet, in which an average pore diameter of the separation substrate is 2.0 μm to 12.0 μm, and the separation substrate is formed of at least one resin selected from the group consisting of a polysulfone resin and a polyvinylidene fluoride resin.

### CROSS-REFERENCE TO RELATED APPLICATIONS

This application is a Continuation of PCT International Application No. PCT/JP2018/015926 filed on Apr. 18, 2018, which claims priority under 35 U.S.C. § 119(a) to Japanese Patent Application No. 2017-095834 filed on May 12, 2017. The above application is hereby expressly incorporated by reference, in its entirety, into the present application.

### BACKGROUND OF THE INVENTION

### 1. Field of the Invention

The present invention relates to a separation substrate, a cell separation filter, and a method for producing a platelet.

### 2. Description of the Related Art

Because platelets play a central role in the formation of blood clots and are cells having a hemostatic function in vivo, in a case where platelets decrease when bleeding occurs or when anticancer drugs are used, death may occur in severe cases.

In addition, the only established treatment for a platelet decrease is to transfuse platelet formulations. At present, platelet formulations rely on blood donation from volunteers, and it is expected that maintaining a balance between supply and demand in the medical field will be difficult because of a decrease in the population of blood-donable age groups due to a declining birthrate and an increase in the population of elderly people with high demand for blood donation, despite 4 days of a storage validity period, which is extremely short.

Accordingly, attention has been focused on the development of platelet sources that can replace blood donations.

In recent years, a technology has been reported for mass production of platelets in vitro by culturing megakaryocytes with use of pluripotent stem cells, hematopoietic precursor cells, mesenchymal lineage cells, and the like as sources.

In this technology, platelets are produced by breaking up the cytoplasm of megakaryocytes, and thereby a culture solution after platelet production contains a large number of megakaryocytes.

For this reason from the viewpoint of suppressing immunogenicity, it is necessary to develop a technology for separating megakaryocytes and platelets produced from the megakaryocytes.

As such a separation technique, for example, JP2016-192960A discloses a “separation substrate composed of a porous material for separating platelets from a cell suspension containing megakaryocytes and platelets, in which in a porous body, an average pore diameter on an inflow side is 10 μm to 20 μm, an average pore diameter decreases continuously or stepwise from the inflow side to an outflow side, and an average pore diameter on the outflow side is 3 μm to 8 μm” (claim 1).

### SUMMARY OF THE INVENTION

The inventors of the present invention have examined a platelet separation substrate disclosed in JP2016-192960A, and have found that a blocking rate (a removal rate) of megakaryocytes was high, but a permeation rate (a recovery rate) of platelets was low, and there is still room for improvement in a separation performance of megakaryocytes and platelets.

An object of the present invention is to provide a separation substrate having a high megakaryocyte blocking rate and a high platelet permeation rate, and a cell separation filter and a method for producing a platelet which use the same.

As a result of intensive studies to achieve the above-mentioned object, the inventors of the present invention have found that, in a case where a separation substrate which made of a porous membrane has an average pore diameter of 2.0 μm to 12.0 μm, and in which a material is composed of a polysulfone resin and/or a polyvinylidene fluoride resin, a megakaryocyte blocking rate becomes high, and a platelet permeation rate becomes high, and therefore have completed the present invention.

That is, it has been found that the above-described object can be achieved with the following configuration.

[1] A separation substrate comprising a porous membrane for separating a platelet from a cell suspension containing a megakaryocyte and the platelet,

in which an average pore diameter of the separation substrate is 2.0 μm to 12.0 μm, and

the separation substrate is formed of at least one resin selected from the group consisting of a polysulfone resin and a polyvinylidene fluoride resin.

[2] The separation substrate according to [1], in which the separation substrate has a pore diameter distribution in which a pore diameter decreases continuously or discontinuously from a surface in a central thickness direction.

[3] The separation substrate according to [1], in which a surface of the separation substrate is modified with a hydrophilic polymer or a hydrophilic group.

[4] A cell separation filter comprising: a container in which a first liquid inlet and a second liquid inlet are disposed; and a filtering medium filled between the first liquid inlet and the second liquid inlet,

in which the filtering medium is the separation substrate according to any one of [1] to [3].

[5] A method for producing a platelet, comprising:

a contact step of contacting the separation substrate according to any one of [1] to [3] with a culture solution containing at least a megakaryocyte;

a culture step of culturing a megakaryocyte to produce a platelet at least before or after the contact step; and

a recovery step of recovering a culture solution containing a produced platelet after the contact step and the culture step.

According to the present invention, it is possible to provide a separation substrate having a high megakaryocyte blocking rate and a high platelet permeation rate, and a cell separation filter and a method for producing a platelet which use the same.

### DESCRIPTION OF THE PREFERRED EMBODIMENTS

Hereinafter, the present invention will be described in detail.

The explanation of the configuration requirements described below is based on representative embodiments of the present invention; however, the present invention is not intended to be limited to such embodiments.

In the present specification, a numerical value range expressed using “to” means a range including numerical values before and after “to” as a lower limit value and an upper limit value.

In general, a separation substrate is a structure having a large number of small voids therein, and examples thereof include a fiber structure, a porous membrane, a bead-filled column, and a laminate of these.

The fiber structure is a structure in which fibers are intertwined, such as woven fabric (mesh), knitted fabric, braided cord, non-woven fabric, and a fiber filled in a column. Among them, non-woven fabric is particularly preferable from the viewpoints of wide pore diameter distribution, complicated flow paths, and ease of production. Examples of methods for producing non-woven fabric include a dry method, a wet method, a spunbond method, a melt blow method, an electrospinning method, a needle punch method, and the like. Among them, from the viewpoint of productivity and versatility, the wet method, the melt blow method, and the electrospinning method are preferable.

A porous membrane has countless communication holes in the entire plastic body. Examples of manufacturing methods therefor include a phase separation method, a foaming method, an etching method of radiating radiant rays and laser light, a porogen method, a freeze-drying method, a plastic sintering method, and the like. A porous membrane obtained by using a phase separation method is particularly preferable from the viewpoint of a wide pore diameter distribution, a complicated flow path, and ease of production.

A column filled with beads is a column in which voids are formed by filling beads in the column. It is desirable that a bead particle diameter is uniform, and it is easy to control the void between the beads as the pore diameter depending on the bead particle diameter.

[Separation Substrate]

The separation substrate of the embodiment of the present invention is a separation substrate including a porous membrane for separating a platelet from a cell suspension containing a megakaryocyte and the platelet.

In addition, an average pore diameter of the separation substrate of the embodiment of the present invention is preferably 2.0 μm to 12.0 μm, and is more preferably 2.0 μm to 9.0 μm.

Furthermore, the separation substrate of the embodiment of the present invention is formed of at least one resin selected from the group consisting of a polysulfone resin and a polyvinylidene fluoride resin, and is preferably formed of at least a polysulfone resin.

In the present specification, the “average pore diameter” is a value obtained by evaluation by increasing air pressure to 2 cc/min with respect to a sample completely wetted by GALWICK (manufactured by Porous, Materials Inc.) in a pore diameter distribution measurement test using a perm porometer (CFE-1200 AEX manufactured by Seika Corporation).

Specifically, with respect to a membrane sample completely wetted by GALWICK, a certain amount of air was fed at 2 cc/min to one side of the membrane, and while measuring a pressure thereof, a flow rate of air permeating to an opposite side of the membrane is measured.

In this method, firstly, data on pressure and permeated air flow rate (hereinafter referred to as a “wet curve”) is obtained for the membrane sample wetted by GALWICK. Next, the same data (hereinafter referred to as a “dry curve”) was measured for the membrane sample in a dry state, and a pressure at an intersection of a curve (half dry curve) corresponding to half of a dry curve flow rate and the wet curve is calculated. Thereafter, an average pore diameter can be calculated by introducing a surface tension (γ) of GALWICK, a contact angle (θ) with the substrate, and an air pressure (P) into Formula (I).

Average pore diameter=4γ cos θ/P (I)

As described above, since the separation substrate of the embodiment of the present invention has an average pore diameter is 2.0 μm to 12.0 μm, and is formed of a polysulfone resin and/or a polyvinylidene fluoride resin, a megakaryocyte blocking rate becomes high and a platelet permeation rate becomes high.

The reason why such effects are exhibited is not elucidated in detail. The inventors of the present invention speculate the reason as follows.

That is, from the comparison of Examples 1 to 3 and Comparative Examples 1 to 4 to be described later, it is considered that, in a case where an average pore diameter of the separation substrate is 2.0 μm to 12.0 μm, permeation of megakaryocytes can be blocked, and platelets can be permeated.

In addition, based on the results of Comparative Examples 5 to 9 to be described later, even in a case where an average pore diameter of the separation substrate is 2.0 μm to 12.0 μm, in a case where the separation substrate is formed of a resin material not corresponding to a polysulfone resin and/or a polyvinylidene fluoride resin, evaluation becomes inferior. Therefore, it is considered that, in the present invention, a polysulfone resin and/or a polyvinylidene fluoride resin forming the separation substrate has properties of easily adsorbing megakaryocytes and hardly adsorbing platelets.

A thickness of the separation substrate of the embodiment of the present invention is preferably 10.0 μm to 500.0 μm, is more preferably 50.0 μm to 500.0 μm, and is even more preferably 100.0 μm to 300.0 μm.

In the present specification, a “thickness” is a value obtained by measuring a membrane thickness of the separation substrate 10 points using a micrometer (manufactured by Mitutoyo) and averaging the respective measured values.

In the present invention, from the reason of further improving a separation performance with respect to megakaryocytes and platelets, the separation substrate preferably has a pore diameter distribution in which a pore diameter decreases continuously or discontinuously from a surface in a central thickness direction.

In the present specification, a “pore diameter distribution” refers to a distribution measured as follows.

First, the separation substrate is impregnated with methanol and frozen in liquid nitrogen.

Next, sections for cross section observation are cut out from the frozen porous separation substrate with a microtome (EM UC6 manufactured by Leica), and are imaged using Scanning Electron Microscope (SEM) (SU8030 type FE-SEM manufactured by Hitachi High-Technologies Corporation). The SEM imaging is performed with a magnification of 3000 times.

Regarding the cutting with a microtome, the separation substrate is divided into 10 sections in a thickness direction from one surface side of the separation substrate, pores of each obtained section are traced with a digitizer, and thereby an average pore diameter of 50 pores in each section is obtained. However, for a section in which pores are large and therefore only 50 or less pores can be measured, only available number of pores that can be counted in that section is measured.

Next, the obtained average pore diameter of each section is plotted in order from one surface to the other surface, and thereby distribution of the average pore diameter in the thickness direction of the membrane is obtained.

In addition, in the present invention, the number average molecular weight (Mn) of a polysulfone resin and/or a polyvinylidene fluoride resin is not particularly limited, and is preferably 1,000 to 10,000,000, and is more preferably 5,000 to 1,000,000.

In the present specification, a “number average molecular weight” is a value measured by a gel permeation chromatography (GPC) method under the following conditions.
Apparatus name: HLC-8220 GPC (Tosoh Corporation)
Type of column: TSK gel Super HZ4000 and HZ2000 (Tosoh Corporation)
Eluent: Dimethylformamide (DMF)
Flow rate: 1 ml/min
Detector: RI
Sample concentration: 0.5%
Calibration curve base resin: TSK standard polystyrene (molecular weights 1,050, 5,970, 18,100, 37,900, 190,000, and 706,000)

In the present invention, for the reason of suppressing adsorption of platelets to the separation substrate and improving a recovery percentage of platelets, all or some of a part of the separation substrate which comes into contact with a cell suspension containing megakaryocytes and platelets is preferably hydrophilized by modifying a hydrophilic polymer or hydrophilic group.

In the present specification, a “hydrophilic polymer” and a “hydrophilic group” respectively refer to a polymer and a functional group capable of making a static contact angle of water of a surface modified using the same 80° or smaller. In addition, the term “modification” refers to a concept including not only the case where a hydrophilic polymer or a hydrophilic group is chemically bonded to the surface of a separation substrate, but also physical adsorption due to hydrophobic interaction or the like.

The hydrophilic polymer is preferably a polymer having a hydrophilic group in the side chain. Examples thereof include 2-methacryloyloxyethyl phosphorylcholine, ethylene glycol, methyl methacrylate, hydroxyethyl methacrylate, vinyl alcohol, N-vinyl-2-pyrrolidone, a polymer of a sulfobetaine monomer, and the like.

In addition, specific examples of hydrophilic groups include a hydroxyl group, an ether group, a nitro group, an imino group, a carbonyl group, a phosphoric acid group, a methoxydiethylene glycol group, a methoxytriethylene glycol group, an ethoxydiethylene glycol group, an ethoxytriethylene glycol group, an amino group, a dimethylamino group, a diethylamino group, a carboxyl group, a phosphoryl group, a phosphorylcholine group, a sulfone group, and salts thereof.

A modification method with a hydrophilic polymer or a hydrophilic group is not particularly limited, and examples thereof include hydrophilic treatments such as plasma treatment, corona treatment, ultraviolet (UV) ozone treatment, flame treatment, and the like. By these treatments, a hydrophilic group such as a hydroxyl group can be introduced on the surface of the separation substrate to make the surface of the separation substrate hydrophilic.

Furthermore, as a hydrophilic polymer, a hydrophilic group, and a modification method thereof, materials and methods disclosed in WO87/005812A, JP1992-152952A (JP-1104-152952A), JP1993-194243A (JP-H05-194243A), WO2010/113632A, and the like can be used.

The separation substrate of the embodiment of the present invention may contain other components as additives in addition to a polysulfone resin and a polyvinylidene fluoride resin.

Specific examples of additives include metal salts of inorganic acids such as common salts, sodium chloride, lithium chloride, sodium nitrate, potassium nitrate, sodium sulfate, and zinc chloride; metal salts of organic acids such as sodium acetate and sodium formate; polymers such as polyethylene glycol and polyvinyl pyrrolidone; high polymer electrolytes such as sodium polystyrene sulfonate and polyvinyl benzyl trimethyl ammonium chloridel; ionic surfactants such as sodium dioctyl sulfosuccinate and sodium alkyl sodium taurate; and the like.

The separation substrate of the embodiment of the present invention may be a porous membrane composed of a plurality of layers, but is preferably a porous membrane composed of a single layer.

[Manufacturing Method]

A method for manufacturing the separation substrate (the porous membrane) of the embodiment of the present invention is not particularly limited, and a general method for forming a polymer membrane can be used.

Examples of methods for forming a polymer membrane include a stretching method, a casting method, and the like. For example, in the casting method, it is possible to produce a porous membrane having the above-mentioned average pore diameter by adjusting the type and amount of a solvent used in a stock solution for forming a membrane, and a drying method after casting.

Manufacture of a porous membrane by a casting method can be carried out by a method including, for example, the following (1) to (4) in this order.

(1) A stock solution for forming a membrane containing a polysulfone resin and/or a polyvinylidene fluoride resin (hereinafter, will be abbreviated as a “polymer” in the description of a method for manufacturing a porous membrane), the above-described additives which may be added as necessary, and any solvent which may be used as necessary, is flow-cast on a support while being in a dissolved state.

(2) The surface of the flow-cast liquid membrane is exposed to temperature-controlled humid air.

(3) The membrane obtained after being exposed to temperature-controlled humid air is immersed in a coagulation liquid.

(4) A support is peeled off if necessary.

A temperature of the temperature-controlled humid air is preferably 4° C. to 60° C., and is more preferably 10° C. to 40° C.

A relative humidity of the temperature-controlled humid air is preferably 30% to 70%, and is more preferably 40% to 50%.

An absolute humidity of the temperature-controlled humid air is preferably 1.2 to 605 g/kg air, and is more preferably 2.4 to 30.0 g/kg air.

The temperature-controlled humid air is preferably applied for 0.1 seconds to 30 seconds, and is more preferably applied for 1 seconds to 10 seconds, at a wind speed of 0.1 m/s to 10 m/s.

An average pore diameter and position of the compact portion can also be controlled by a moisture concentration contained in the temperature-controlled humid air and a time of applying the temperature-controlled humid air. An average pore diameter of the compact portion can also be controlled by an amount of moisture contained in a stock solution for forming a membrane.

By applying the temperature-controlled humid air to the surface of the liquid membrane as described above, it is possible to cause coacervation from the surface of the liquid membrane toward the inside by controlling evaporation of a solvent.

In this state, by immersing the membrane in a coagulation liquid containing a solvent having low solubility of the polymer but is compatible with the solvent used for the stock solution for forming a membrane, the above-mentioned coacervation phase is fixed as fine pores, and pores other than the fine pores can also be formed.

A temperature of the coagulating liquid is preferably −10° C. to 80° C. in a process of immersing the membrane in the coagulating liquid. By changing a temperature during this period, it is possible to control a size of a pore diameter up to a support surface side by adjusting a time from the formation of the coacervation phase on the support surface side to the solidification from the compact portion.

In a case where a temperature of the coagulating liquid is raised, the formation of the coacervation phase becomes faster and a time for solidification becomes longer, and therefore the pore diameter toward the support surface side tends to become large. On the other hand, in a case where a temperature of the coagulating liquid is lowered, the formation of the coacervation phase becomes slower and a time for solidification becomes shorter, and therefore the pore diameter toward the support surface side is unlikely to become large.

As the support, a plastic film or a glass plate may be used. Examples of materials of the plastic film include polyester such as polyethylene terephthalate (PET); polycarbonate; acrylic resin; epoxy resin; polyurethane; polyamide; polyolefin; cellulose derivatives; silicone; and the like.

As the support, PET or a glass plate is preferable, and PET is more preferable.

The stock solution for forming a membrane may contain a solvent. A solvent having high solubility of the polymer to be used (hereinafter will be abbreviated as a “favorable solvent”) may be used depending on a polymer to be used.

As a favorable solvent, it is preferable that the solvent be quickly substituted with the coagulation liquid in a case where the membrane is immersed in the coagulation liquid.

Examples of solvents include N-methyl-2-pyrrolidone, dioxane, tetrahydrofuran, dimethylformamide, dimethylacetamide, or a mixed solvent thereof in a case where the polymer is polysulfone resin; and include N-methyl-2-pyrrolidone, tetrahydrofuran, dimethylformamide, dimethylacetamide, tetramethylurea, dimethyl sulfoxide, trimethyl phosphate, or a mixed solvent thereof in a case where the polymer is a polyvinylidene fluoride resin.

In addition to a favorable solvent, the stock solution for forming a membrane preferably uses a solvent (hereinafter will be abbreviated as a “non-solvent”) in which the solubility of the polymer is low but is compatible with the solvent of the polymer.

Examples of non-solvents include water, cellosolves, methanol, ethanol, propanol, acetone, tetrahydrofuran, polyethylene glycol, glycerin, and the like. Among them, it is preferable to use water.

A concentration of the polymer as the stock solution for forming a membrane is preferably 5% by mass to 35% by mass, and is more preferably 10% by mass to 30% by mass.

By setting the concentration of the polymer to 35 mass % or less, sufficient permeability can be imparted to the obtained porous membrane. By setting the concentration thereof to 5 mass % or more, the formation of a porous membrane which selectively allows substances to permeate can be secured.

In addition, an additional amount of the optional additives described above is not particularly limited as long as uniformity of the stock solution for forming a membrane is not lost by the addition, but is 0.5% by volume to 10% by volume respect to a general solvent.

Furthermore, in a case where the stock solution for forming a membrane contains a non-solvent and a favorable solvent, a ratio of the non-solvent to the favorable solvent is not particularly limited as long as a mixed solution can be maintained in a homogeneous state, but is preferably 1.0% by mass to 50% by mass, is more preferably 2.0% by mass to 30% by mass, and is even more preferably 3.0% by mass to 10% by mass.

As the coagulation liquid, it is preferable to use a solvent having a low solubility of the polymer used.

Examples of such solvents include water, alcohols such as methanol, ethanol, and butanol; glycols such as ethylene glycol and diethylene glycol; aliphatic hydrocarbons such as ether, n-hexane, and n-heptane; glycerol such as glycerin; and the like.

Examples of preferable coagulation liquids include water, alcohols, or a mixture of two or more of these. Among them, it is preferable to use water.

After immersion in the coagulation liquid, it is also preferable to perform washing with a solvent different from the coagulation liquid that has been used.

Washing can be carried out by immersion in a solvent.

Diethylene glycol is preferable as a washing solvent. Distribution of an N element in the porous membrane can be adjusted by adjusting either or both of a temperature and an immersion time of diethylene glycol in which a film is immersed by using diethylene glycol as a washing solvent. In particular, in a case where polyvinylpyrrolidone is used as an additive in the stock solution for forming a membrane of the porous membrane, a residual amount of polyvinylpyrrolidone on the membrane can be controlled. After washing with diethylene glycol, furthermore, the membrane may be washed with water.

As the stock solution for forming a membrane of the porous membrane, the stock solution for forming a membrane, which is obtained by dissolving polysulfone resin and polyvinylpyrrolidone in N-methyl-2-pyrrolidone and adding water, is preferable.

Regarding a method for manufacturing the porous membrane, reference can be made to JP1992-349927A (JP-H04-349927A), JP1992-068966B (JP-H04-068966B), JP1992-351645A (JP-H04-351645A), JP2010-235808A, and the like.

[Cell Suspension]

The cell suspension used for the separation of platelets using the separation substrate of the embodiment of the present invention is a cell suspension containing megakaryocytes and platelets.

Megakaryocytes and platelets are not particularly limited, and examples thereof include megakaryocytes and platelets collected from adult tissue; megakaryocytes and platelets differentiated from cells having differentiation ability such as pluripotent stem cells, hematopoietic precursor cells, and mesenchymal cells; megakaryocytes and platelets produced by using direct reprogramming techniques on cells that do not have the ability to differentiate into megakaryocytes by methods of the related art; megakaryocytes and platelets combining these; and the like.

Examples of pluripotent stem cells include embryonic stem cells ((ES) cells), nuclear transfer embryonic stem cells ((nt) ES cells), and induced pluripotent stem cells ((iPS) cells), and the like. Among them, induced pluripotent stem cells (iPS cells) are preferable.

Examples of hematopoietic precursor cells include cells derived from bone marrow, cells derived from umbilical cord blood, cells derived from (granulocyte-colony stimulating factor: G-CSF)-mobilized peripheral blood, middle lobe lung cells derived from ES cells, cells derived from peripheral blood, and the like, but are not limited thereto. Examples of these hematopoietic precursor cells include cells positive to cluster of differentiation (CD) 34 (for example, CD34+ cells, CD133+ cells, SP cells, CD34+CD38− cells, c-kit+ cells, or CD3−, CD4−, CD8−, and CD34+ cells) (WO2004/110139A).

Examples of mesenchymal cells include mesenchymal stem cells, adipose precursor cells, bone marrow cells, adipocytes and synovial cells, among which adipose precursor cells are preferable.

Examples of cells that do not have an ability to be differentiated into megakaryocytes by general methods include fibroblasts and the like, but are not limited thereto.

[Cell Separation Filter]

The cell separation filter of the embodiment of the present invention is a cell separation filter including a container in which a first liquid inlet and a second liquid inlet are disposed; and a filtering medium filled between the first liquid inlet and the second liquid inlet, in which the separation substrate of the embodiment of the present invention described above is used for the filtering medium.

A form, size, and material of the container used for the cell separation filter are not particularly limited.

A form of the container may be any form such as, for example, a sphere, a container, a cassette, a bag, a tube, or a column.

As the container type, any of a cross flow type and a column type can be used.

[Method for Producing Platelet]

The method for producing a platelet of the embodiment of the present invention includes a contact step of contacting the separation substrate of the embodiment of the present invention described above with a culture solution containing at least a megakaryocyte;

a culture step of culturing a megakaryocyte to produce a platelet before and/or after the contact step; and

a recovery step of recovering a culture solution containing a produced platelet after the contact step and the culture step.

A contact means in the contact step can be appropriately selected according to the amount of the culture solution and the concentration of megakaryocytes. Examples thereof include a method of supplying a cell suspension to a tower or column filled with the separation substrate of the embodiment of the present invention.

In addition, examples of means for producing platelets in the culture step include a method of applying a shear stress by a fluid, and specifically, a method of stirring a culture solution containing megakaryocytes. Furthermore, the megakaryocyte cultured in the culture step may be a megakaryocyte supplemented with the separation substrate of the embodiment of the present invention, in a case where the culture step is performed after the contact step. Furthermore, in a case where the culture step is performed after the contact step, as will be described later in examples, it is considered that in a case where the cell suspension containing megakaryocytes and platelets is brought into contact with the separation substrate, platelets are produced in megakaryocytes captured in the initial stage even by loading due to the cell suspension (for example, a fluid) that comes into contact therewith.

Furthermore, examples of recovery means in the recovery step include a method of passing a culture solution containing the produced platelets through a column or column filled with the separation substrate of the embodiment of the present invention.

### EXAMPLES

Hereinafter, the present invention will be described in more detail based on Examples. The materials, amounts used, proportions, treatments, treatment procedures, and the like disclosed in the following Examples can be modified as appropriate as long as the gist of the present invention is maintained. Therefore, the scope of the present invention should not be limitedly interpreted by the Examples described below.

### Example 1

<Porous Membrane>

15 parts by mass of polysulfone resin (P3500, manufactured by Amoco), 15 parts by mass of polyvinylpyrrolidone, 2 parts by mass of lithium chloride, and 1.2 parts by mass of water were dissolved in 66.8 parts by mass of N-methyl-2-pyrrolidone. Therefore, a mixture for forming a membrane was obtained.

This mixture was flow-cast on a surface of a PET film by a thickness of 200 μm.

Next, the flow-cast membrane surface was exposed to air adjusted to 25° C. and absolute humidity 7.8 g/kg air, at 2 m/sec for 5 seconds.

Immediately thereafter, the exposed membrane surface was immersed in a coagulation liquid tank at a temperature of 40° C. which is filled with water.

Next, after the PET was peeled off, at 2 m/sec, the immersed membrane surface was put into a diethylene glycol bath at 25° C. for 120 seconds, and then was sufficiently washed with pure water. Thereby, a porous membrane was produced.

<Megakaryocytes and Platelets>

Medium: A medium in which 50 ml of bovine serum (Life Technologies) was added to 450 ml of RPMI1640 (Life Technologies) was used.

Megakaryocyte: MEG-01 (ATCC) was used as the megakaryocyte. A megakaryocyte liquid (6×10⁵ cells/ml) was prepared by mixing this with a medium.

Platelet suspension: A platelet suspension isolated from rat peripheral blood was used as platelets. Specifically, 10 ml of whole blood collected from a rat was recovered in a 15 ml conical tube for centrifugation (manufactured by Falcon) which contains a citrate-dextrose solution (ACD) (manufactured by Sigma-Aldrich). Centrifugation was performed at 300×g and room temperature for 7 minutes, and the plasma layer and the Buffy coat layer after centrifugation were recovered. The recovered solution was centrifuged in the same manner, and only the Plasma layer was recovered, and then centrifuged at 1800×g at room temperature for 5 minutes, and the supernatant was recovered to obtain platelets. This was mixed with a medium to prepare a platelet suspension (6×10⁷ cells/ml).

A cell suspension was prepared by mixing equal volumes of megakaryocyte fluid and platelet suspension.

<Cell Separation Test>

Membrane separation treatment was performed using a filtration module in which one flow port on the supply side of a filtration module (ADVANTEC, KS-47) was connected to a 50 ml syringe (Terumo) containing a cell suspension. The syringe was placed in a syringe pump (HARVARD APPARATUS, PHD ULTRA 4400), and the syringe pump was operated so that 30 ml of the cell suspension was supplied at a flow rate of 3 ml/min in a dead-end manner that goes straight to the separation substrate installed in the filtration module. The filtrate discharged from the permeate side outlet of the filtration module was recovered.

<Count of the Number of Recovered Cells>

100 μl of filtrate collected from the permeate side of the filtration module was added to 10 μl of Dulbecco's Phosphate-Buffered Saline (DPBS) (manufactured by Thermo Fisher Scientific) in which Hoechst 33342 (manufactured by Dojindo Laboratories), which is a nuclear stain, was added, and the mixture was reacted for 15 minutes in a light-shielded environment. 300 μl of DPBS was added thereto, and measurement was performed by flow cytometry (FACS Aria) by using BD Trucount tubes (manufactured by Nippon Becton, Dickinson and Company).

Megakaryocyte fraction and platelet fraction were determined from forward scatter (FSC) and side scatter (SSC) gates. Nuclear stain negative cells in the platelet fractions are perceived as platelets, and nuclear stain positive cells in the megakaryocyte fractions are perceived as megakaryocytes. Therefore, the number of platelets and the number of megakaryocytes in the recovered solution was calculated.

A platelet permeation rate and a megakaryocyte blocking rate obtained from the following equation are shown in Table 1.

Platelet permeation rate (%)=(the number of platelets in filtrate/the number of platelets in original solution)×100

Megakaryocyte blocking rate (%)=100−(number of megakaryocytes in filtrate/number of megakaryocytes in original solution)×100

<Evaluation (Separation Judgment)>

As a general judgment of separation, the following criteria were evaluated. The results are shown in Table 1.

A: A platelet permeation rate is 80% or more, and a megakaryocyte blocking rate is 95% or more.

B: A platelet permeation rate is 80% or more, and a megakaryocyte blocking rate is 90% or more.

Alternatively, a platelet permeation rate is 70% or more, and a megakaryocyte blocking rate is 95% or more.

C: A platelet permeation rate is less than 70%, or a megakaryocyte blocking rate is less than 90%.

### Example 2 and Comparative Examples 1 to 3

A separation substrate was produced and evaluated in the same manner as in Example 1 except that a porous membrane having an average pore diameter and a thickness shown in Table 1 was produced while changing a moisture concentration contained in temperature-controlled humid air, and a time to expose the porous membrane to the temperature-controlled humid air when producing the porous membrane. The results are shown in Table 1.

### Example 3

Evaluation was performed in the same manner as in Example 1 using a porous membrane made of hydrophilic polyvinylidene fluoride (SVLP04700, manufactured by Merck Millipore). The results are shown in Table 1.

### Comparative Example 4

Evaluation was performed in the same manner as in Example 1 using a porous membrane made of hydrophilic polyvinylidene fluoride (DVPP04700, manufactured by Merck Millipore). The results are shown in Table 1.

### Comparative Example 5

Evaluation was performed in the same manner as in Example 1 using a porous membrane made of hydrophilic polytetrafluoroethylene (manufactured by Merck Millipore). The results are shown in Table 1.

### Comparative Example 6

Evaluation was performed in the same manner as in Example 1 using a porous membrane made of polycarbonate (manufactured by Merck Millipore). The results are shown in Table 1.

### Comparative Example 7

Evaluation was performed in the same manner as in Example 1 using a porous membrane made of cellulose acetate (manufactured by ADVANTEC). The results are shown in Table 1.

### Comparative Example 8

Evaluation was performed in the same manner as in Example 1 using a porous membrane made of cellulose acetate/nitrocellulose (A300A047A, manufactured by ADVANTEC). The results are shown in Table 1.

### Comparative Example 9

Evaluation was performed in the same manner as in Example 1 using a porous membrane made of cellulose acetate/nitrocellulose (A500A047A, manufactured by ADVANTEC). The results are shown in Table 1.

TABLE 1

| | | Pore diameter | Average | | Result of separation test | | |
|---|---|---|---|---|---|---|---|
| | | distribution | pore | | Platelet | | |
| | | (thickness | diameter | Thickness | permeation | Megakaryocyte | |
| | Material | direction) | (μm) | (μm) | rate | blocking rate | Evaluation |
| Example 1 | Polysulfone resin | Distribution | 2.9 | 153 | 85.8% | 99.9% | A |
| Example 2 | Polysulfone resin | Distribution | 8.9 | 174 | 91.2% | 99.1% | A |
| Example 3 | Hydrophilic | No | 2.8 | 105 | 79.0% | 99.9% | B |
| | polyvinylidene fluoride | distribution | | | | | |
| Comparative | Polysulfone resin | Distribution | 0.8 | 171 | 1.8% | 99.8% | C |
| Example 1 | | | | | | | |
| Comparative | Polysulfone resin | Distribution | 1.4 | 168 | 3.6% | 99.8% | C |
| Example 2 | | | | | | | |
| Comparative | Polysulfone resin | Distribution | 13.0 | 163 | 99.2% | 78.5% | C |
| Example 3 | | | | | | | |
| Comparative | Hydrophilic | No | 0.7 | 105 | 1.7% | 99.9% | C |
| Example 4 | polyvinylidene fluoride | distribution | | | | | |
| Comparative | Hydrophilic | No | 2.9 | 28 | 7.5% | 99.9% | C |
| Example 5 | polytetrafluoroethylene | distribution | | | | | |
| Comparative | Polycarbonate | No | 4.4 | 22 | 92.8% | 89.2% | C |
| Example 6 | | distribution | | | | | |
| Comparative | Cellulose acetate | No | 3.0 | 89 | 37.2% | 99.9% | C |
| Example 7 | | distribution | | | | | |
| Comparative | Cellulose | No | 2.1 | 89 | 4.6% | 99.9% | C |
| Example 8 | acetate/nitrocellulose | distribution | | | | | |
| Comparative | Cellulose | No | 3.3 | 88 | 10.7% | 99.8% | C |
| Example 9 | acetate/nitrocellulose | distribution | | | | | |

Based on the results shown in Table 1, it was found that, in a case where a separation substrate made of a porous membrane having an average pore diameter of less than 2.0 μm was used, the platelet permeation rate becomes low (Comparative Examples 1, 2, and 4).

In addition, it was found that, in a case where a separation substrate made of a porous membrane having an average pore diameter larger than 12.0 μm was used, a blocking rate of megakaryocytes becomes low (Comparative Example 3).

Furthermore, it was found that, even in a case where an average pore diameter of the separation substrate is 2.0 μm to 12.0 μm, in a case where a material of the separation substrate is not composed of at least one resin selected from the group consisting of a polysulfone resin and a polyvinylidene fluoride resin, any one of a platelet permeation rate or the megakaryocyte blocking rate becomes low (Comparative Examples 5 to 9).

In contrast, it was found that, in a case where an average pore diameter of the separation substrate is 2.0 μm to 12.0 μm, and a material of the separation substrate is composed of at least one resin selected from the group consisting of a polysulfone resin and a polyvinylidene fluoride resin, the megakaryocyte blocking rate becomes high, and the platelet permeation rate becomes high (Examples 1 to 3).

What is claimed is:

1. A separation substrate comprising:
a porous membrane for separating a platelet from a cell suspension containing a megakaryocyte and the platelet,
wherein an average pore diameter of the separation substrate is 2.0 μm to 12.0 μm, and
the separation substrate is formed of at least one resin selected from the group consisting of a polysulfone resin and a polyvinylidene fluoride resin.

2. The separation substrate according to claim 1, wherein the separation substrate has a pore diameter distribution in which a pore diameter decreases continuously or discontinuously from a surface in a central thickness direction.

3. The separation substrate according to claim 1, wherein a surface of the separation substrate is modified with a hydrophilic polymer or a hydrophilic group.

4. A cell separation filter comprising:
a container in which a first liquid inlet and a second liquid inlet are disposed; and
a filtering medium filled between the first liquid inlet and the second liquid inlet, wherein the filtering medium is the separation substrate according to claim 1.

5. A cell separation filter comprising:
a container in which a first liquid inlet and a second liquid inlet are disposed; and
a filtering medium filled between the first liquid inlet and the second liquid inlet,
wherein the filtering medium is the separation substrate according to claim 2.

6. A cell separation filter comprising:
a container in which a first liquid inlet and a second liquid inlet are disposed; and
a filtering medium filled between the first liquid inlet and the second liquid inlet,
wherein the filtering medium is the separation substrate according to claim 3.

7. A method for producing a platelet, comprising:
a contact step of contacting the separation substrate according to claim 1 with a culture solution containing at least a megakaryocyte;
a culture step of culturing a megakaryocyte to produce a platelet at least before or after the contact step; and
a recovery step of recovering a culture solution containing a produced platelet after the contact step and the culture step.

8. A method for producing a platelet, comprising:
a contact step of contacting the separation substrate according to claim 2 with a culture solution containing at least a megakaryocyte;
a culture step of culturing a megakaryocyte to produce a platelet at least before or after the contact step; and
a recovery step of recovering a culture solution containing a produced platelet after the contact step and the culture step.

9. A method for producing a platelet, comprising:
a contact step of contacting the separation substrate according to claim 3 with a culture solution containing at least a megakaryocyte;
a culture step of culturing a megakaryocyte to produce a platelet at least before or after the contact step; and
a recovery step of recovering a culture solution containing a produced platelet after the contact step and the culture step.
